(19) United States
(12) Patent Application Publication
(10) Pub. No.: US 2009/0298848 A1
(43) Pub. Date: Dec. 3, 2009

(54) Methods and Compositions for Treating Barth Syndrome, Cardiomyopathy, Mitochondrial Diseases and Other Conditions

(75) Inventor: Timothy Andrew Stewart, San Francisco, CA (US)

Correspondence Address: CANTOR COLBURN, LLP, 20 Church Street, 22nd Floor, Hartford, CT, 06103, US

(73) Assignee: CORTENDO INVEST AB,, Halmstad 302 34 (SE)

(21) Appl. No.: 12/297,356

(22) PCT Filed: Apr. 18, 2007
(86) PCT No.: PCT/US07/66892

§ 371 Date: Jan. 5, 2009

(30) Foreign Application Priority Data
Apr. 18, 2006 (US) 60793308

(51) Int. Cl. A61K 31/497

(52) U.S. Cl. 514/254.07

(57) ABSTRACT

Pharmaceutical compositions comprising the 2S,4R ketoconazole enantiomer or its pharmaceutically acceptable salts, hydrates, and solvates are useful to increase cardiolipin synthesis and for the treatment of Barth Syndrome, diabetic myopathy, cardiomyopathy associated with aging, mitochondrial disease, and other conditions and disorders where cardiolipin deficiency plays a causative or symptomatic role.

### CROSS REFERENCE TO RELATED APPLICATIONS

This application claims priority to U.S. provisional application No. 60/793,308 (filed Apr. 18, 2006) the entire content of which is incorporated herein by reference.

### FIELD OF THE INVENTION

The present invention relates to methods for treating diseases that can be treated by increasing cardiolipin synthesis, including but not limited to cardiomyopathy associated with Barth Syndrome, diabetes, ageing and mitochondrial diseases. The invention therefore relates to the fields of chemistry, biology, pharmacology, and medicine.

### BACKGROUND OF THE INVENTION

Ketoconazole, 1-acetyl-4-[4-[[2-(2,4-dichlorophenyl)-2-[(1H-imidazol-1-yl)-methyl]-1,3-dioxolan-4-yl]methoxy]phenyl]piperazine, is a racemic mixture of the cis enantiomers (−)-(2S,4R) and (+)-(2R,4S) marketed as an anti-fungal agent. Ketoconazole inhibits fungal growth through the inhibition of ergosterol synthesis. Ergosterol is a key component of fungal cell walls.

More recently, ketoconazole was found to decrease plasma cortisol and to be useful, alone and in combination with other agents, in the treatment of a variety of diseases and conditions, including type 2 diabetes, Metabolic Syndrome (also known as the Insulin Resistance Syndrome, Dysmetabolic Syndrome or Syndrome X), and other medical conditions that are associated with elevated cortisol levels. See U.S. Pat. Nos. 6,166,017; 6,642,236; and 6,881,739, each of which is incorporated herein by reference.

Ketoconazole has also been reported to lower cholesterol levels in humans (Sonino et al. (1991). “Ketoconazole treatment in Cushing's syndrome: experience in 34 patients.” Clin Endocrinol (Oxf). 35(4): 347-52; Gylling et al. (1993). “Effects of ketoconazole on cholesterol precursors and low density lipoprotein kinetics in hypercholesterolemia.” J Lipid Res. 34(1): 59-67), each of which is incorporated herein by reference).

Cardiolipin is a diphosphatidyl glycerol. The majority of the cardiolipin molecules contain four linoleic acid molecules. This lipid is a key component of the mitochondrial inner membrane and serves to stabilize the electron transport chain. Defects in the synthesis of cardiolipin cause Barth Syndrome (Hauff and Hatch 2006). Children with Barth Syndrome develop severe and fatal cardiomyopathy. There is no effective therapy for patients with Barth Syndrome. Patients with diabetes also develop cardiomyopathy (left ventricular systolic and diastolic dysfunction, left ventricular hypertrophy, and alterations in the coronary microcirculation). Diabetic cardiomyopathy is associated with decreased cardiolipin (Han, Yang et al. 2005). While there are an increasing number of therapeutic options for the treatment of the hyperglycemia associated with diabetes, heart disease is still a leading cause of death in patients with diabetes. Cardiolipin levels decrease with ageing and cardiomyopathy rates increase with age.

Thus, there remains a need for new methods for treating diseases and conditions associated with decreased cardiolipin levels or activity or that may be treated by increasing cardiolipin level or activity. The present invention meets these and other needs.

### SUMMARY OF THE INVENTION

The present invention arises in part from the discovery that ketoconazole and, more specifically, the 2S,4R enantiomer of ketoconazole increases cardiolipin levels.

The present invention provides methods for treating diseases and conditions associated with decreased cardiolipin levels, production rates or activity and other diseases and conditions that can be treated by increasing cardiolipin levels, production rates or activity, by administering a pharmaceutical composition containing a therapeutically effective amount of the 2S,4R ketoconazole enantiomer. In one embodiment, racemic ketoconazole, containing both the 2S,4R and 2R,4S enantiomers is employed in the methods of the invention. In another embodiment, the 2S,4R enantiomer, substantially free of the 2R,4S enantiomer, is employed in the methods of the invention.

### BRIEF DESCRIPTION OF THE FIGURES

FIG. 1 shows the effect of the ketoconazole 2S,4R enantiomer on hepatic cardiolipin. The figure shows that the 2S,4R enantiomer is able to increase the levels of cardiolipin that contain both saturated and unsaturated fatty acids. Male Beagle dogs were treated with empty gelatin capsules (Placebo) or gelatin capsules containing sufficient 2S,4R enantiomer of ketoconazole to provide a dose of 20 mg/kg body weight. After 91 days of therapy, the amount of cardiolipin present in the livers of the dogs was determined. The amount of cardiolipin with fatty acids of the different indicated classes was determined independently. SFA, saturated fatty acids; MUFA, mono-saturated fatty acids; PUFA, poly-unsaturated fatty acid; n3, fatty acids in which the first double bond is at C3; n6, fatty acids in which the first double bond is at C6; n9, fatty acids in which the first double bond is at C9.

FIG. 2 shows the effect of the ketoconazole 2S,4R enantiomer on hepatic cardiolipin. The figure shows that the 2S,4R enantiomer is able to increase the levels of cardiolipin that contain fatty acids of a wide variety of carbon chain length with different numbers of carbon bonds. Male Beagle dogs were treated with empty gelatin capsules (Placebo) or gelatin capsules containing sufficient 2S,4R enantiomer of ketoconazole to provide a dose of 20 mg/kg body weight. After 91 days of therapy, the amount of cardiolipin present in the livers of the dogs was determined. The amount of cardiolipin with fatty acids of the different indicated classes was determined independently. The different classes are arranged according to the proportion that they are found in the placebo treated animals.

### DETAILED DESCRIPTION OF THE INVENTION

The present invention provides methods for treating diseases and conditions associated with decreased cardiolipin levels and diseases and conditions that may be medically treated by increasing cardiolipin levels. To aid in understanding the invention, this detailed description is organized as follows. Section I describes compositions useful in the methods of the invention, as well as methods for preparing the ketoconazole 2S,4R enantiomer, its solvates and salts, and pharmaceutical compositions comprising it. Section II describes unit dosage forms of the pharmaceutical compositions of the invention and methods for administering them. Section III describes methods for treating diseases and conditions by administration of ketoconazole or the 2S,4R ketoconazole enantiomer and pharmaceutical compositions comprising the 2S,4R ketoconazole enantiomer.

### I. Preparation of the 2S,4R Ketoconazole Enantiomer and Pharmaceutical Compositions Containing the 2S,4R Ketoconazole Enantiomer

In one embodiment, the methods of the invention can be practiced by administering racemic ketoconazole. In this embodiment, commercially available compositions may be employed at the appropriate dose levels or methods known in the art can be used to prepare such compositions. Racemic ketoconazole formulated for oral administration is commercially available and approved for the treatment of fungal infections. In another embodiment, the methods of the invention can be practiced by administering the 2S,4R ketoconazole enantiomer in a pharmaceutical formulation substantially free of the 2R,4S enantiomer. As used herein, a composition containing the 2S,4R ketoconazole enantiomer includes compositions that do not contain the 2R,4S ketoconazole enantiomer as well as compositions that contain substantially less of the 2R,4S ketoconazole enantiomer, relative to the amount of the 2S,4R enantiomer, than do racemic ketoconazole, as well as compositions that contain the 2R,4S enantiomer at levels equal to or greater than the 2S,4R enantiomer. Racemic ketoconazole is a composition containing both the 2S,4R and 2R,4S enantiomers.

The 2S,4R enantiomer of ketoconazole may be obtained by optical resolution of racemic ketoconazole. Such resolution can be accomplished by any of a number of resolution methods well known to a person skilled in the art, including but not limited to those described in Jacques et al., “Enantiomers, Racemates and Resolutions,” Wiley, New York (1981), incorporated herein by reference. For example, the resolution may be carried out by preparative chromatography on a chiral column. Another example of a suitable resolution method is the formation of diastereomeric salts with a chiral acid such as tartaric, malic, mandelic acid or N-acetyl derivatives of amino acids, such as N-acetyl leucine, followed by recrystallization to isolate the diastereomeric salt of the desired enantiomer. Yet another method for obtaining compositions of the 2S,4R enantiomer substantially free of the 2R,4S enantiomer is a fractional crystallization of the diastereomeric salt of ketoconazole with (+)-camphor-10-sulfonic acid.

The 2S,4R enantiomer of ketoconazole can also be prepared directly by a variety of methods known to those of skill in the art. For example, the 2S,4R enantiomer can be prepared directly by transketolization reactions between 2-bromo-2′,4′-dichloroacetophenone and optically pure solketal tosylates, as described by Rotstein et al. (“Stereoisomers of ketoconazole: preparation and biological activity.” J Med Chem 1992; 35(15): 2818-25, incorporated herein by reference).

The methods of the present invention can be practiced with a variety of pharmaceutically acceptable salts of the 2S,4R enantiomer of ketoconazole. As used herein the term “pharmaceutically acceptable salt of the 2S,4R enantiomer of ketoconazole” includes mixtures of the 2S,4R and the 2R,4S enantiomers of ketoconazole. The term “pharmaceutically acceptable salt” refers to salts prepared from pharmaceutically acceptable bases or acids, including inorganic or organic bases and inorganic or organic acids. Salts derived from inorganic bases include aluminum, ammonium, calcium, copper, ferric, ferrous, lithium, magnesium, manganic, manganous, potassium, sodium, and zinc salts, and the like. The ammonium, calcium, magnesium, potassium, and sodium salts, in particular, can be preferred for some pharmaceutical formulations. Salts in the solid form can exist in more than one crystal structure and can also be in the form of hydrates and polyhydrates. The solvates, and, in particular, the hydrates of the 2S,4R ketoconazole enantiomer are useful in the preparation of pharmaceutical compositions useful in the methods of the present invention.

Salts derived from pharmaceutically acceptable organic bases include salts of primary, secondary and tertiary amines, substituted amines, including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and tromethamine, and the like.

When the compound to be formulated is basic, salts can be prepared from pharmaceutically acceptable acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acid, and the like. Illustrative pharmaceutically acceptable acids include citric, hydrobromic, hydrochloric, maleic, phosphoric, sulfuric, and tartaric acids. Ketoconazole compounds are often basic, because the triazole ring is basic. The 2S,4R ketoconazole compound can be made and handled as a non-pharmaceutically acceptable salt (e.g. trifluoroacetate salts) during synthesis and then converted as described herein to a pharmaceutically acceptable salt.

Suitable pharmaceutically acceptable salts of the 2S,4R ketoconazole enantiomer include, but are not limited to, the mesylate, maleate, fumarate, tartrate, hydrochloride, hydrobromide, esylate, p-toluenesulfonate, benzoate, acetate, phosphate, and sulfate salts. For the preparation of pharmaceutically acceptable acid addition salts of the compound of 2S,4R ketoconazole, the free base can be reacted with the desired acids in the presence of a suitable solvent by conventional methods. Similarly, an acid addition salt can be converted to the free base form by methods known to those of skill in the art.

Pharmaceutical compositions useful in the methods of the invention can contain as the active pharmaceutical ingredient metabolites of the 2S,4R ketoconazole enantiomer that are therapeutically active or prodrugs of the enantiomer. Prodrugs are compounds that are converted to therapeutically active compounds as they are being administered to a patient or after they have been administered to a patient.

Thus, the pharmaceutical compositions useful in the methods of the invention comprise the 2S,4R ketoconazole enantiomer, or a pharmaceutically acceptable salt, hydrate or solvate thereof, or a prodrug or active metabolite thereof, in combination with a pharmaceutically acceptable carrier. In one embodiment, the pharmaceutical composition contains a therapeutically effective amount of the 2S,4R enantiomer of ketoconazole or a pharmaceutically acceptable salt thereof and a pharmaceutically acceptable carrier. As noted above, pharmaceutically acceptable salts of the 2S,4R enantiomer useful in such compositions include, but are not limited to, the hydrochloride, phosphate, maleate, fumarate, tartrate, mesylate, esylate, and sulfate salts.

The “therapeutically effective amount” of the 2S,4R enantiomer of ketoconazole or pharmaceutically acceptable salt thereof will depend on the condition to be treated, the route and duration of administration, the physical attributes of the patient, including weight and other medications taken concurrently, and may be determined according to methods well known to those skilled in the art in light of the present disclosure (see Section II, below). The pharmaceutical compositions useful in the methods of the invention can be conveniently prepared in unit dosage form by methods well-known in the art of pharmacy as medicaments to be administered orally, parenterally (including subcutaneous, intramuscular, and intravenous administration), ocularly (ophthalmic administration), rectally, pulmonarily (nasal or oral inhalation), topically, transdermally or via buccal transfer.

The pharmaceutical compositions useful in the methods of the invention can be prepared by combining the 2S,4R ketoconazole enantiomer with a selected pharmaceutical carrier according to conventional pharmaceutical compounding techniques. Carriers take a wide variety of forms. For example, carriers for oral liquid compositions include, e.g., water, glycols, oils, alcohols, flavoring agents, preservatives, coloring agents and other components used in the manufacture of oral liquid suspensions, elixirs and solutions. Carriers such as starches, sugars and microcrystalline cellulose, diluents, granulating agents, lubricants, binders, disintegrating agents and the like are used to prepare oral solid dosage forms, e.g., powders, hard and soft capsules and tablets. Solid oral preparations are typically preferred over oral liquid preparations.

Thus, in one embodiment, the pharmaceutically acceptable carrier is a solid and the pharmaceutical composition is a tablet for oral administration. Other suitable forms of the pharmaceutical compositions useful in the methods of the invention for oral administration include compressed or coated pills, dragees, sachets, hard or soft gelatin capsules, sublingual tablets, syrups and suspensions. The oral solid dosage forms may also contain a binder such as gum tragacanth, acacia, corn starch, or gelatin; excipients such as dicalcium phosphate; a disintegrating agent such as corn starch, potato starch, or alginic acid; a lubricant such as magnesium stearate; and/or a sweetening agent such as sucrose, lactose, or saccharin. Capsules may also contain a liquid carrier such as a fatty oil. Various other materials may be present to act as coatings or to modify the physical form of the dosage unit. For instance, tablets may be coated with shellac, sugar or both. Tablets may be coated by standard aqueous or nonaqueous techniques. The typical percentage of active compound in these compositions may, of course, be varied from, for example and without limitation, about 2 percent to about 60 percent on a w/w basis.

In another embodiment, the pharmaceutically acceptable carrier is a liquid, and the pharmaceutical composition is intended for oral administration. Oral liquids suitable for use in such compositions include syrups and elixirs and can contain, in addition to the active ingredient, sucrose as a sweetening agent, methyl and propylparabens as preservatives, a dye, and/or a flavoring, such as cherry or orange flavor.

In another embodiment, the methods of the present invention are practiced by administering a pharmaceutical composition of the 2S,4R ketoconazole enantiomer suitable for parenteral administration. For parenteral administration, the pharmaceutical composition is typically contained in ampoules or vials and consists essentially of an aqueous or non-aqueous solution or emulsion. These compositions are typically in the form of a solution or suspension, and are typically prepared with water, and optionally include a surfactant such as hydroxypropylcellulose. Dispersions can be prepared in glycerol, liquid polyethylene glycols, and mixtures thereof in oils. Typically, preparations that are in diluted form also contain a preservative.

In another embodiment, the pharmaceutically acceptable carrier is a liquid, and the pharmaceutical composition is an injectable solution. The pharmaceutical injectable dosage forms, including aqueous solutions and dispersions and powders for the extemporaneous preparation of injectable solutions or dispersions, are also sterile and, at the time of administration, are sufficiently fluid for easy syringability. These compositions are stable under the conditions of manufacture and storage and are typically preserved. The carrier thus includes the solvent or dispersion medium containing, for example, water, ethanol, polyol (e.g. glycerol, propylene glycol and liquid polyethylene glycol), suitable mixtures thereof, and vegetable oils.

In another embodiment, the pharmaceutically acceptable carrier is a gel, and the pharmaceutical composition is provided in the form of a suppository. For rectal administration, the pharmaceutical composition is provided in a suppository, and the pharmaceutical acceptable carrier is a hydrophilic or hydrophobic vehicle. In another embodiment, the pharmaceutical composition useful in the methods of the invention is prepared for topical application, and the 2S,4R ketoconazole enantiomer is formulated as an ointment. The 2S,4R enantiomer can also be administered transdermally; suitable transdermal delivery systems are known in the art.

The pharmaceutical compositions of the invention also include sustained release compositions. Suitable sustained release compositions include those described in U.S. patent application publication Nos. 20050013834; 20030190357; and 2002055512 and PCT patent application publication Nos. WO 03011258 and 0152833, each of which is incorporated herein by reference.

### II. Unit Dosage Forms; Frequency and Duration of Administration

As noted above, any suitable route of administration can be employed for providing a mammal, typically a human, but mammals of veterinary importance, such as cattle, horses, pigs, sheep, dogs, and cats, can also benefit from the methods described herein, with a therapeutically effective dose of the 2S,4R enantiomer. For example, oral, rectal, topical, parenteral, ocular, pulmonary, or nasal administration can be employed. Dosage forms include tablets, troches, dispersions, suspensions, solutions, capsules, creams, ointments, aerosols and the like. In many embodiments of the treatment methods of the invention, the pharmaceutical composition is administered orally. The therapeutically effective dosage of the active ingredient varies depending on the particular compound employed (salt, solvate, prodrug, or metabolite), the mode of administration, the condition being treated, and the severity of the condition. Such dosages may be ascertained readily by a person skilled in the art in light of the disclosure herein.

When treating or preventing the diseases and conditions as described herein, satisfactory results can obtained when the 2S,4R ketoconazole enantiomer is administered at a daily dosage of from about 0.1 to about 25 milligrams (mg) per kilogram (mpk) of body weight, preferably given as a single daily dose or in divided doses about two to six times a day. For oral administration to a human adult patient, the therapeutically effective amount will generally be administered in the range of 50 mg to 750 mg per dose, including but not limited to 150 mg per dose, 300 mg per dose, and 450 mg per dose, and multiple, usually consecutive daily doses will be administered in a course of treatment. The 2S,4R ketoconazole enantiomer-containing pharmaceutical composition can be administered at different times of the day. In one embodiment the optimal therapeutic dose can be administered in the evening. In another embodiment the optimal therapeutic dose can be administered in the morning. The total daily dosage of the 2S,4R ketoconazole enantiomer thus can in one embodiment range from about 10 mg to about 2 g, and often ranges from about 10 mg to about 1 g, and most often ranges from about 100 mg to about 500 mg. In the case of a typical 70 kg adult human, the total daily dose of the 2S,4R ketoconazole enantiomer can range from about 10 mg to about 1000 mg and will often range, as noted above, from about 50 mg to about 750 mg. This dosage may be adjusted to provide the optimal therapeutic response.

In those embodiments of the invention in which racemic ketoconazole is administered, the amount administered is the amount that contains the therapeutically effective amount of the 2S,4R enantiomer specified in the preceding paragraph. Typically, for an adult human, the total daily dose of racemic ketoconazole in accordance with the methods of the present invention will range from about 10 mg to about 1000 mg.

In one embodiment, the unit dosage form is suitable for oral administration and contains one or more pharmaceutical excipients. Examples of pharmacologically inactive excipients that can be included in an orally available formulation of the 2S,4R enantiomer of ketoconazole for purposes of the present invention and their function are provided in the following table.

| Inactive Ingredient | Trade Name | Grade | Function |
|---|---|---|---|
| Silicified | Prosolv HD 90 | NF | Diluent |
| Microcrystalline | | | |
| Cellulose | | | |
| Lactose Monohydrate | Modified, 316 Fast Flo | NF | Diluent |
| Corn Starch | STA-Rx | NF | Disintegrant |
| Magnesium Stearate | N/A | NF | Lubricant |
| Colloidal Silicon | Cab-O-Sil M5P | NF | Glidant |
| Dioxide | | | |

The excipients listed in the preceding table can be combined in varying proportion with the 2S,4R enantiomer to obtain specific drug tablet and manufacturing characteristics. The drug tablet size can vary from 1 mg total weight to 1000 mg total weight; for example and without limitation, from 100 mg total weight to 800 mg total weight. The proportion of the 2S,4R enantiomer present in the drug tablet can vary from 1% to 100%; for example and without limitation, from 10% to 90%. An example of a 300 mg tablet with the 2S,4R enantiomer comprising 50% of the tablet weight is provided in the following table. In this example, dry blends were made with the (−) cis 2S,4R ketoconazole and the listed inactive excipients and pressed as a dry blend into tablets.

| | | Tablet |
|---|---|---|
| | | Weight |
| Component | % w/w | (mg) |
| (−)cis 2S,4R Ketoconazole | 50.0 | 150 |
| Lactose Monohydrate, NF | 22.4 | 67.2 |
| Silicified Microcrystalline Cellulose, NF | 16.5 | 49.5 |
| Corn Starch, NF | 10.0 | 30.0 |
| Colloidal Silicon Dioxide, NF) | 0.5 | 1.5 |
| Magnesium Stearate, NF | 0.6 | 1.8 |
| Total | 100.0 | 300.0 |

A drug tablet formulation for 2S,4R ketoconazole was described in U.S. Pat. No. 6,040,307. This formulation included the active drug substance, (−) ketoconazole, Lactose, Cornstarch, water and Magnesium Stearate. Wet granules were generated with the ketoconazole, lactose, water and corn starch, these granules were dried in an oven prior to compressing into tablets with magnesium stearate and more corn starch. Tablets were compressed and dried. This is a less optimal method than the method described above using a dry blend process, as excess water and elevated temperatures are not introduced. Ketoconazole can undergo degradation (oxidation) (Farhadi and Maleki (2001). “A new spectrophotometric method for the determination of ketoconazole based on the oxidation reactions.” Analytical Sciences 17 Supplement, i867-i869. The Japan Society for Analytical Chemistry), and oxidation reactions are accelerated in the presence of water and elevated temperatures.

The solid unit dosage forms of the pharmaceutical compositions employed in the methods of the invention contain the 2S,4R ketoconazole enantiomer or a salt or hydrate thereof in an amount ranging from about 1 mg to about 2 g, often from about 1.0 mg to about 1.0 g, and more often from about 10 mg to about 500 mg. In the liquid pharmaceutical compositions employed in the methods of the invention suitable for oral administration, the amount of the 2S,4R ketoconazole enantiomer can range from about 1 mg/ml to about 200 mg/ml. The therapeutically effective amount can also be an amount ranging from about 10 mg/ml to about 100 mg/ml. In one embodiment, the dose of the liquid pharmaceutical composition administered is an amount between 0.5 ml and 5.0 ml. In another embodiment, the dose is between about 1 ml and 3 ml. In the liquid pharmaceutical compositions for use in the methods of the invention designed for intravenous or subcutaneous administration, the amount of the 2S,4R ketoconazole can range from about 0.01 to 1 mg/ml and can be administered at a rate of between 0.01 to 1 ml/minute by either a subcutaneous or intravenous administration. Alternatively the amount of the 2S,4R enantiomer can range from about 0.1 mg/ml to 10 mg/ml and can be administered at a rate of between 0.001 ml/minute to 0.1 ml/minute by either of a subcutaneous or intravenous administration.

As noted above, the pharmaceutical compositions employed in the methods of the invention will typically be administered for multiple consecutive days for periods ranging from one or more weeks to one, several, or many months. In one embodiment, the pharmaceutical compositions employed in the methods of the invention are administered for the treatment of a chronic disease, condition, or indication for treatment periods ranging from one month to twelve months. In another embodiment, the 2S,4R enantiomer is administered from one year to five years. In another embodiment, the 2S,4R enantiomer is administered from 5 years to 20 years. In another embodiment, the 2S,4R enantiomer is administered until there is remission from the disease or for the life of the patient.

The duration of administration in accordance with the methods of the invention depends on the disease or condition to be treated, the extent to which administration of the pharmaceutical composition has ameliorated the disease symptoms and conditions, and the individual patient's reaction to the treatment.

III. Methods for Treating Diseases and Conditions Associated with Decreased Cardiolipin Levels and/or Activity

### Cardiolipin

Diphosphatidylglycerol or cardiolipin (1,3-bis(sn-3′-phosphatidyl)-sn-glycerol) is a dimeric structure, having four acyl groups. In eukaryotes, it is found only in membranes of mitochondria, subcellular organelles whose function is to generate an electrochemical potential for substrate transport and ATP synthesis. It amounts to about 10% of the phospholipids of bovine heart muscle, and 20% of the phospholipids of the mitochondrial membrane. The biosynthetic pathway to diphosphatidylglycerol is similar to that of other phospholipids in that it passes through the common intermediates, phosphatidic acid and phosphatidyl-CMP. However, the final step is a unique reaction. Eukaryotic diphosphatidylglycerol synthase links an activated phosphatidyl moiety (phosphatidyl-CMP) to phosphatidylglycerol. In eukaryotes, cardiolipin is the only phospholipid synthesised in the mitochondrion, and it remains there for the life of the cell. In animal tissues, diphosphatidylglycerol contains almost exclusively 18 carbon fatty acids, and 80% of this is typically linoleic acid (18:2 (n−6)).

As diphosphatidylglycerol is the specific lipid component of mitochondria, its biological function in this organelle is clearly crucial. It is located mainly on the inner membrane of mitochondria, where it interacts with a large number of mitochondrial proteins. This interaction effects functional activation of certain enzymes, especially those involved in oxidative phosphorylation.

The present invention provides methods for using the 2S,4R enantiomer of ketoconazole for the treatment, control, amelioration, prevention, delay in the onset of or reduction of the risk of developing the diseases and conditions due at least in part to reduced cardiolipin levels in a mammalian patient, particularly a human. In one embodiment, the method involves the administration of a therapeutically effective amount of the 2S,4R ketoconazole enantiomer or a pharmaceutically acceptable salt or solvate thereof, or a racemic mixture containing the 2S,4R and 2R,4S enantiomers, to the patient suffering from the disease or condition.

Decreased cardiolipin can contribute to a large number of diseases and conditions, including, but not limited to, Barth Syndrome, diabetic cardiomyopathy, cardiomyopathies associated with aging and mitochondrial diseases including but not limited to MELAS (Myopathy, encephalopathy, lactic acidosis, and stroke-like episodes). These and other diseases and conditions susceptible to treatment with the methods of the invention are described below.

### Barth Syndrome

Barth syndrome (BTHS), a human disease state (cardiomyopathy) linked to the X-chromosome, is associated with marked abnormalities in the fatty acid composition of cardiolipin, i.e. a decrease in tetralinoleoyl molecular species, and an accumulation of monolysocardiolipin. The cardiomyopathy associated with BTHS is mainly of the dilated type. Cardiac dysfunction in BTHS usually presents in the first year of life, and may present as early as the first day of life. A variable, mainly left-sided, ventricular and septal hypertrophy often coexists with ventricular dilation. Endocardial fibroelastosis, dilated cardiomyopathy, and left ventricular dilation/hypertrophy may occur in the same individuals. Sudden ventricular tachycardia may lead to cardiac arrest and death. Skeletal muscle weakness is also a characteristic finding in BTHS. Exertional fatigue is the combined result of cardiomyopathy and skeletal myopathy. Weakness is usually present early in life.

Mutations that introduced stop codons in the gene TAZ G4.5 have been found in BTHS families. The derived proteins have been termed tafazzins. Mutation studies in a number of families established a variety of mutations including frameshifts by 1-2 basepair insertions or deletions, as well as non-sense, splice-site, and missense mutations. In one large family, a frameshift mutation causing a stop codon in exon 8 lead to death within the first months of life in all affected boys. A mutation at this site affects all mRNA transcripts. The TAZ gene is critical for the remodeling of PG, and mutations in this gene can result in a deficiency of tetralinoleoyl-cardiolipin (L4-CL) in myocardial tissue from both right and left ventricle and skeletal muscle.

The muscular (cardiac and skeletal) abnormalities in patients with Barth Syndrome are caused by decreased synthesis of cardiolipin. The 2S,4R enantiomer and pharmaceutical compositions containing this enantiomer, including but not limited to those approved for the treatment of fungal infection, can act to increase the amount of cardiolipin. There remains a need for new methods of treating Barth Syndrome (BTHS). The present invention meets this need. The present invention provides a method of treating BTHS in a mammalian patient in need of such treatment, which method comprises administering to said patient a therapeutically effective amount of a pharmaceutical composition containing the 2S,4R enantiomer of ketoconazole. Administration of a therapeutically effective amount of a compound such as the 2S,4R ketoconazole enantiomer that can increase cardiolipin levels is effective in treating, controlling, and ameliorating the symptoms of BTHS and administration of a therapeutically effective amount of a compound such as the 2S,4R ketoconazole enantiomer that is able to increase cardiolipin levels on a regular, daily basis can delay or prevent the development of BTHS.

### Diabetic Cardiomyopathy

Diabetic cardiomyopathy is characterized by diastolic dysfunction, and/or with myocardial ischemia and left ventricular hypertrophy. Left ventricular hypertrophy is also more prevalent in type 2 diabetic patients and contributes to ventricular dysfunction. While therapeutic use of ACE inhibitors is of benefit in these patients, diabetic cardiomyopathy is still a major cause of mortality and morbidity in patients with diabetes, and there is a need for improved therapeutic options. Cardiolipin levels are decreased in cardiac myopathy (see Han, X., J. Yang, et al. (2005). “Shotgun lipidomics identifies cardiolipin depletion in diabetic myocardium linking altered substrate utilization with mitochondrial dysfunction.” Biochemistry 44(50): 16684-94) and could cause cardiac dysfunction through increased levels of cardio-toxic FFA oxidation products.

Increased levels of cardiolipin are beneficial in treating or controlling diabetic myopathy. In one embodiment, the invention provides a method of treating diabetic myopathy in a mammalian patient in need of such treatment, said method comprising administering to said patient a therapeutically effective amount of a pharmaceutical composition containing the 2S,4R ketoconazole enantiomer.

Cardiomyopathy Associated with Ageing

Various cardiomyopathies increase with aging. These structural and functional pathologies include hypertrophic cardiomyopathy, idiopathic dilated cardiomyopathy, left atrial enlargement, left ventricular hypertrophy and congestive heart failure. As cardiac cardiolipin decreases with aging (see Paradies, G., F. M. Ruggiero, et al. (1992). “The effect of aging and acetyl-L-carnitine on the activity of the phosphate carrier and on the phospholipid composition in rat heart mitochondria.” Biochim Biophys Acta 1103(2): 324-6; and Lee, H. J., J. Mayette, et al. (2006). “Selective remodeling of cardiolipin fatty acids in the aged rat heart.” Lipids Health Dis 5: 2), and as decreased cardiac cardiolipin will cause cardiopathies, the administration of a therapeutically effective amount of a compound such as the 2S,4R ketoconazole enantiomer that will increase cardiolipin levels can be beneficial in treating, or reducing the severity of cardiomyopathy associated with aging. In one embodiment, the invention provides a method of treating a cardiomyopathy in a patient in need of such treatment, said method comprising administering to said patient a therapeutically effective amount of a pharmaceutical composition containing the 2S,4R ketoconazole enantiomer.

### Mitochondrial Diseases

Mitochondrial cytopathies or mitochondrial diseases represent a heterogeneous group of multisystem disorders which preferentially affect the muscle and nervous systems and are caused by defective oxidative phosphorylation (OXPHOS). They have an incidence of 1 in 11,000 children, and also have a high prevalence in adults. In addition to mutations in the mitochondrial genome, several nuclear genes associated with mtDNA maintenance have been also found to be associated with mitochondrial disorders. The ubiquitous distribution of the mitochondria in the human body explains the multiple organ involvement. To date, the treatment of these diseases remains supportive and includes administration of antioxidants (vitamin E, alpha lipoic acid), electron donors and acceptors (coenzyme Q 10, riboflavin), alternative energy sources (creatine monohydrate), lactate reduction strategies (dichloroacetate) and exercise training. As decreased cardiolipin causes mitochondrial disfunction and skeletal and neural abnormalities in patients with BTHS and as deficiencies in cardiolipin have been described in patients with mitochondrial disease (see Schlame, M., S. Shanske, et al. (1999). “Microanalysis of cardiolipin in small biopsies including skeletal muscle from patients with mitochondrial disease.” J Lipid Res 40(9): 1585-92), the administration of a therapeutically effective amount of a compound such as the 2S,4R ketoconazole enantiomer that will increase cardiolipin levels can be beneficial in treating and reducing the severity of mitochondrial diseases. In one embodiment, the invention provides a method of treating mitochondrial disease in a patient in need of such treatment, said method comprising administering to said patient a therapeutically effective amount of a pharmaceutical composition containing the 2S,4R ketoconazole enantiomer.

In view of the foregoing, those of skill in the art will appreciate that the present invention provides a method of treating a condition selected from the group consisting of: (1) Barth Syndrome, (2) diabetic myopathy, (3) cardiomyopathy associated with ageing, (4) mitochondrial disease and (5) other conditions and disorders where cardiolipin deficiency is a component, in a mammalian patient in need of such treatment, said method comprising administering to the patient a therapeutically effective amount of a pharmaceutical composition of the 2S,4R ketoconazole enantiomer.

In another aspect, the present invention provides a method of delaying the onset of a condition selected from the group consisting of (1) Barth Syndrome, (2) diabetic myopathy, (3) cardiomyopathy associated with aging, (4) mitochondrial disease and (5) other conditions and disorders where cardiolipin deficiency is a component in a mammalian patient in need of such treatment, said method comprising administering to the patient a therapeutically effective amount of a pharmaceutical composition of the 2S,4R ketoconazole enantiomer.

In another aspect, the present invention provides a method of reducing the risk of developing a condition selected from the group consisting of (1) Barth Syndrome, (2) diabetic myopathy, (3) cardiomyopathy associated with ageing, (4) mitochondrial disease and (5) other conditions and disorders where cardiolipin deficiency is a component in a mammalian patient in need of such treatment, said method comprising administering to the patient a therapeutically effective amount of a pharmaceutical composition of the 2S,4R ketoconazole enantiomer.

In some embodiments, the patient to whom the 2S,4R ketoconazole enantiomer is administered is diabetic and has been diagnosed as having diabetic cardiomyopathy. In some embodiments the patient to whom the 2S,4R ketoconazole enantiomer is administered is diabetic and has a blood sugar level controlled by administration of insulin, an insulin analog, an insulin substitute or other blood glucose level controlling agent. In some embodiments the patient to whom the 2S,4R ketoconazole enantiomer is administered is diabetic and has not been administered the 2S,4R ketoconazole enantiomer prior to being diagnosed as in need of treatment for cardiomyopathy. In some embodiments the patient to whom the 2S,4R ketoconazole enantiomer is administered is not diabetic and has been diagnosed as in need of treatment for cardiomyopathy.

In some embodiments, a subjects's cardiolipin level may be determined before, during, and/or after administration of the 2S,4R ketoconazole enantiomer. Cardiolipin levels can be determined using any quantitative or qualitative assay (for illustration and not limitation see Ritov et al., 2006, Analysis of cardiolipin in human muscle biopsy. J Chromatogr B Analyt Technol Biomed Life Sci. 831:63-71; Gomez and Robinson, 1999, Quantitative determination of cardiolipin in mitochondrial electron transferring complexes by silicic acid high-performance liquid chromatography. Anal Biochem. 267:212-6; Schlame et al., 1999, Microanalysis of cardiolipin in small biopsies including skeletal muscle from patients with mitochondrial disease. J Lipid Res. 40:1585-92; Schlame et al., 2002, Deficiency of tetralinoleoyl-cardiolipin in Barth syndrome. Ann Neurol. 51:634-7). Cardiolipin levels can be assessed prior to drug administration to determine whether a patient is likely to benefit from the cardiolipin-related activity of the 2S,4R ketoconazole enantiomer and/or after or during drug administration to assess the patient's response to the drug.

### Combination Therapies

Thus, a variety of diseases, disorders, and conditions can be treated, controlled, prevented or delayed with the pharmaceutical compositions and methods of this invention, including but not limited to: (1) Barth Syndrome, (2) diabetic myopathy, (3) cardiomyopathy associated with ageing, (4) mitochondrial disease and (5) other conditions and disorders where cardiolipin deficiency is a component. In one embodiment, a method of the invention is practiced on a patient who concurrently receives another treatment for one or more of these conditions.

The pharmaceutical compositions of the invention can be co-administered or otherwise used in combination with one or more other drugs in the treatment, prevention, suppression, or amelioration of the diseases, disorders, and conditions described herein as susceptible to therapeutic intervention in accordance with the methods of the invention. Such other drug(s) may be administered by a route and in an amount commonly used contemporaneously or sequentially with a pharmaceutical composition of the 2S,4R ketoconazole enantiomer. When a pharmaceutical composition of the 2S,4R ketoconazole enantiomer is used contemporaneously with one or more other drugs, a combination product containing such other drug(s) and the 2S,4R ketoconazole enantiomer can be utilized if the two active drugs can be coformulated. Combination therapy in accordance with the methods of the invention also includes therapies in which the pharmaceutical compositions useful in the methods of the invention and one or more other drugs are administered on different overlapping schedules. It is contemplated that, when used in combination with other active ingredients, the pharmaceutical compositions useful in the methods of the present invention or the other active ingredient or both may be used effectively in lower doses than when each is used alone. Accordingly, the pharmaceutical compositions useful in the methods of the present invention include those that contain one or more other active ingredients, in addition to the 2S,4R ketoconazole enantiomer.

Examples of other drugs that may be administered in combination with a pharmaceutical composition of the present invention, either separately or, in some instances, the same pharmaceutical composition, include, but are not limited to: diuretics, adrenergic blocking agents, vasodilators, calcium channel blockers, renin inhibitors, angiotensin converting enzyme (ACE) inhibitors, angiotensin II antagonists, potassium channel activators, other cardiovascular agents, antioxidants (vitamin E, alpha lipoic acid), electron donors and acceptors (coenzyme Q10, riboflavin), alternative energy sources (creatine monohydrate), lactate reduction strategies (dichloroacetate).

Representative diuretics include hydrochlorothiazide, chlorothiazide, acetazolamide, amiloride, bumetamide, benzthiazide, ethacrynic acid, furosemide, indacrinone, metolazone, spironolactone, triamterene, chlorthalidone and the like and pharmaceutically acceptable salts thereof.

Representative adrenergic blocking agents include phentolamine, phenoxybenzamine, prazosin, terazosin, tolazine, atenolol, metoprolol, nadolol, propranolol, timolol, carteolol and the like and pharmaceutically acceptable salts thereof.

Representative vasodilators include hydralazine, minoxidil, diazoxide, nitroprusside and the like and pharmaceutically acceptable salts thereof.

Representative calcium channel blockers include amrinone, bencyclane, diltiazem, fendiline, flunarizine, nicardipine, nimodipine, perhexilene, verapamil, gallopamil, nifedipine and the like and pharmaceutically acceptable salts thereof.

Representative renin inhibitors include enalkiren, zankiren, RO 42-5892, PD-134672 and the like and pharmaceutically acceptable salts thereof.

Representative angiotensin II antagonists include DUP 753, A-81988 and the like.

Representative ACE inhibitors include captopril, enalapril, lisinopril and the like and pharmaceutically acceptable salts thereof.

Representative potassium channel activators include pinacidil and the like and pharmaceutically acceptable salts thereof.

Other representative cardiovascular agents suitable for use in the combination therapies of the invention include sympatholytic agents such as methyldopa, clonidine, guanabenz, reserpine and the like and pharmaceutically acceptable salts thereof.

The compounds of the invention and the cardiovascular agent can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents.

When administered as a combination, the therapeutic agents can be formulated as separate compositions which are given at the same time or different times, or the therapeutic agents can be given as a single composition.

The foregoing is merely illustrative of the invention and is not intended to limit the invention to the disclosed compounds, processes, compositions and methods. Variations and changes which are obvious to one skilled in the art are intended to be within the scope and nature of the invention which are defined in the appended claims.

Thus, in one embodiment, the present invention provides a pharmaceutical composition that comprises: (1) a therapeutically effective amount of 2S,4R ketoconazole enantiomer; (2) a therapeutically effective amount of compound selected from the group consisting of: independently selected from diuretics, adrenergic blocking agents, vasodilators, calcium channel blockers, renin inhibitors, angiotensin converting enzyme (ACE) inhibitors, angiotensin II antagonists, potassium channel activators, other cardiovascular agents, antioxidants (vitamin E, alpha lipoic acid), electron donors and acceptors (coenzyme Q10, riboflavin), alternative energy sources (creatine monohydrate), lactate reduction strategies (dichloroacetate) and (3) a pharmaceutically acceptable carrier.

The above pharmaceutical compositions and combination therapies include those in which the 2S,4R ketoconazole enantiomer, or a pharmaceutically acceptable salt, hydrate, or solvate thereof, is co-formulated or co-administered with one or more other active compounds. Non-limiting examples include combinations of the 2S,4R ketoconazole enantiomer with two or more active compounds selected from diuretics, adrenergic blocking agents, vasodilators, calcium channel blockers, renin inhibitors, angiotensin converting enzyme (ACE) inhibitors, angiotensin II antagonists, potassium channel activators, other cardiovascular agents, antioxidants (vitamin E, alpha lipoic acid), electron donors and acceptors (coenzyme Q10, riboflavin), alternative energy sources (creatine monohydrate), lactate reduction strategies (dichloroacetate).

Thus, in one embodiment, the present invention provides a method of treating a condition selected from the group consisting (1) Barth Syndrome, (2) diabetic myopathy, (3) cardiomyopathy associated with ageing, (4) mitochondrial disease and (5) other conditions and disorders where cardiolipin deficiency is a component, in a mammalian patient in need of such treatment, said method comprising administering to the patient therapeutically effective amounts of a pharmaceutical composition of the 2S,4R ketoconazole enantiomer and of a compound or pharmaceutical composition comprising said compound selected from the group consisting of: diuretics, adrenergic blocking agents, vasodilators, calcium channel blockers, renin inhibitors, angiotensin converting enzyme (ACE) inhibitors, angiotensin II antagonists, potassium channel activators, other cardiovascular agents, antioxidants (vitamin E, alpha lipoic acid), electron donors and acceptors (coenzyme Q10, riboflavin), alternative energy sources (creatine monohydrate), lactate reduction strategies (dichloroacetate).

The following examples illustrate that the 2S,4R enantiomer can increase cardiolipin levels in an animal model and provide a protocol for a clinical trial to demonstrate that the 2S,4R enantiomer can increase cardiolipin levels in humans.

### EXAMPLES

### Example 1

### Measurement of Cardiolipin Following Dosing with the 2S,4R Enantiomer of Ketoconazole

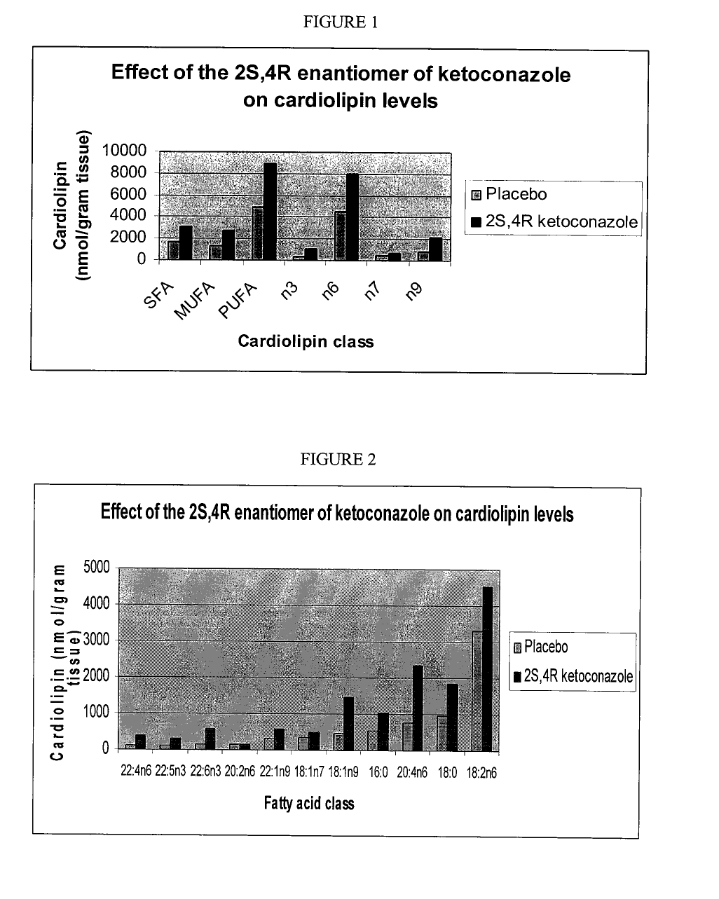

The effect of the 2S,4R enantiomer of ketoconazole enantiomers on cardiolipin levels in Beagle dogs was determined. The dogs were approximately 6-7 months of age at the initiation of the experiment and weighed 8-10 kg. To generate the results shown in FIGS. 1 and 2, eight male beagle dogs were used. Four of these dogs were dosed daily with an empty capsule, and four dogs were dosed with a gelatin capsule containing sufficient 2S,4R enantiomer of ketoconazole for each dog to receive 20 mg/kg body weight of the 2S,4R enantiomer of ketoconazole. The capsules were prepared weekly, placed in a labeled pill bottle and stored at 23° C.±3° C. until dispensed for dosing. The animals were housed throughout the study in suspended stainless steel cages and fed PMI Nutrition International Certified Canine Diet® #5007 during the study as a daily ration. An approximately 400 gram ration of feed was provided daily to each dog beginning on the day after receipt. Municipal tap water following treatment by reverse osmosis was available ad libitum to each animal via an automatic watering device. Environmental controls were maintained at temperatures of 72° F.±7° F. (22° C.±4° C.) with a relative humidity of 50%±20%. A 12-hour light/12-hour dark cycle was maintained, except when interrupted to accommodate study procedures. Ten or greater air changes per hour with 100% fresh air (no air recirculation) was maintained in the animal rooms. Each dog was dosed with either of the empty gelatin capsule or the capsule containing the 2S,4R enantiomer every day for 91 days. After 91 days of daily dosing, the dogs were sacrificed, and the livers of the sacrificed dogs were removed and snap frozen in liquid nitrogen. Lipid content of the livers was determined using a combination of high pressure liquid chromatography (HPLC) and mass spectrometry. The values presented in FIG. 1 represent the amount of cardiolipin in the livers of either the placebo or the 2S,4R enantiomer treated dogs as a function of the liver wet weight. The results demonstrate that the 2S,4R enantiomer increased cardiolipin levels in the treated dogs.

### Example 2

### Formulation and Clinical Trial of the 2S,4R Enantiomer in Type 2 Diabetes

### A. Abbreviations

The following abbreviations are used in this Example.

| Term/ | |
|---|---|
| Abbreviation | Explanation |
| ALT | alanine transaminase |
| AST | aspartate transaminase |
| AUC | area under the curve |
| Bid | twice daily |
| Biw | twice weekly |
| BUN | blood urea nitrogen |
| CV | coefficient of variation |
| ELISA | enzyme-linked immunosorbent assay |
| FDA | Food and Drug Administration |
| GI | Gastrointestinal |
| GLP | Good Laboratory Practice |
| IND | Investigational New Drug (application) |
| IV | Intravenous |
| MedDRA | Medical Dictionary for Regulatory Activities |
| NDA | New Drug Application |
| NOAEL | no-observed-adverse-effect level |
| PBS | phosphate-buffered saline |
| Qd | Daily |
| Qw | Weekly |
| RP-HPLC | reverse-phase high-performance liquid chromatography |
| SBA | Summary Basis of Approval |
| SC | subcutaneous, subcutaneously |
| SD | standard deviation |
| SDS-PAGE | sodium dodecyl sulfate-polyacrylamide gel electrophoresis |
| SE-HPLC | size-exclusion high-performance liquid chromatography |
| USP | United States Pharmacopoeia |
| WBC | white blood cell |

### B. Overview

An illustrative formulation of the 2S,4R enantiomer of ketoconazole is described in this Example together with pre-clinical data supporting its testing as an investigational new drug in human clinical trials. Racemic ketoconazole (the mixture of the two enantiomers 2S,4R and 2R,4S) is an approved drug (NIZORAL®) for the treatment of a variety of fungal infections. As racemic ketoconazole also inhibits cortisol synthesis, this drug is used as a non-approved therapy for patients with Cushing's syndrome. In these patients racemic ketoconazole reduces glucose, cholesterol, and blood pressure. Racemic ketoconazole has, however, been associated with hepatotoxicity. Preclinical results support that the 2S,4R enantiomer of ketoconazole (2S,4R cis-1-acetyl-4-[4-[[2-(2,4-dichlorophenyl)-2-(1H-imidazol-1-ylmethyl)-1,3-dioxolan-4-yl]methoxyl]phenyl]piperazine), also called DIO-902, may be safer and more efficacious than the racemic mixture.

DIO-902 has been purified from the ketoconazole racemic mixture and is largely (greater than 99%) free of the 2R,4S enantiomer. It is anticipated that the primary pharmacological effect of DIO-902 relevant to the treatment of Barth's syndrome will be through the elevation of cardiolipin levels. Secondary benefits of this drug candidate are expected to include reduced total and LDL cholesterol, reduced blood pressure and reduced visceral adiposity.

DIO-902 has been formulated into immediate release tablets. The toxicology of DIO-902 has been tested in dogs. At oral doses of up to 20 mg/kg/day for 28 days the only noted effect was a reduction in food intake and a reduction in body weight and a trend to a decrease in cholesterol. There were no noted changes in any of the other serum chemistry or the hematological parameters measured. Higher single doses have been used in rats. At 200 mg drug/kg body weight DIO-902 suppresses testosterone to 10% of basal. The suppression occurs within four hours of dosing and testosterone levels return to normal within 8 hours. DIO-902 is orally available and reaches a maximal plasma concentration between 2 and 8 hours in dogs. DIO-902 at 200 mg drug/kg body weight reduces serum levels of the active glucocorticoid in rodents (corticosterone) to 25% of basal within 4 hours of oral dosing. This dose of drug also suppresses plasma cholesterol. Thus, DIO-902 (2S,4R) is significantly more potent with respect to reducing corticosterone in rats than is the other enantiomer (2R,4S) and is more potent with respect to reducing cholesterol in rats than is the other enantiomer.

DIO-902 has not been previously administered as a single chemical entity to human patients. DIO-902 has been described as useful in the treatment of other diseases; see PCT patent application No. PCT/US07/00588 and PCT publication WO WO06072881A1, each of which is incorporated herein by reference. However, the active pharmaceutical ingredient of this composition, the 2S,4R enantiomer of ketoconazole, has been widely administered as part of the approved racemic ketoconazole mixture. When normal volunteers are given the racemic mixture, both enantiomers are orally available, and, after a 200 mg dose, a maximum plasma concentration of the DIO-902 (approximately 3.6 μg/mL) is reached at 2 hours. The approved use for the racemic mixture is for the treatment of fungal infections and the approved dose is 200 mg BID. In addition, higher doses of the racemic mixture (up to 2000 mg/day) have been used. In various embodiments of the present invention, racemic ketoconazole can be administered at these doses (200 mg BID and 2000 mg/day). The racemic mixture has also been used for non-approved indications, including Cushing's syndrome and prostate cancer. The racemic mixture can cause hepatoxicity and reduces testosterone, and 1,25 dihydroxy Vitamin D. Because DIO-902 has a reduced inhibitory potency toward CYP7A (the key enzyme in bile acid synthesis and bile formation), DIO-902 is expected to be significantly safer than the approved racemic mixture.

Furthermore DIO-902 has a 12× higher IC₅₀ toward CYP7A (IC₅₀=2.4 microM) than does the 2R,4S enantiomer (IC₅₀=0.195 microM) (Rotstein, Kertesz et al. 1992). CYP7A suppression can lead to functional cholestasis and as a consequence there can be hepatic and plasma accumulation of potentially toxic metabolites such as oxysterols and bilirubin and xenobiotics such as ketoconazole itself. Thus, DIO-902 will be significantly safer than racemic ketoconazole.

### C. Physical Chemical, and Pharmaceutical Properties of an Illustrative Pharmaceutical Formulation of the Invention—DIO 902

DIO-902 is the single enantiomer 2S,4R ketoconazole and is derived from racemic ketoconazole. It may be formulated using cellulose, lactose, cornstarch, colloidal silicon dioxide and magnesium stearate as an immediate release 200 mg or 300 mg strength tablet. The chemical name is 2S,4R cis-1-acetyl-4-[4-[[2-(2,4-dichlorophenyl)-2-(1H-imidazol-1-ylmethyl)-1,3-dioxolan-4-yl]methoxyl]phenyl]piperazine, the formula is C₂₆H₂₈Cl₂N₄O₄, and the molecular weight is 531.44. The CAS number is 65277-42-1, and the structural formula is provided below. The chiral centers are at the carbon atoms 2 and 4 as marked.

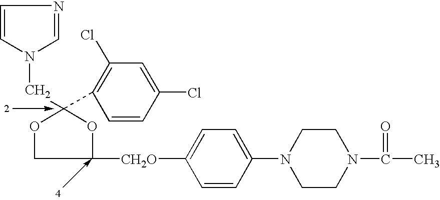

Ketoconazole is an imidazole-containing fungistatic compound. DIO-902 is an immediate release tablet to be taken orally and formulated as shown in the table below.

| Component | Percentage |
|---|---|
| 2S,4R ketoconazole; DIO-902 | 50% |
| Silicified Microcrystalline Cellulose, NF | 16.5 |
| (Prosolv HD 90) | |
| Lactose Monohydrate, NF (316 Fast-Flo) | 22.4 |
| Corn Starch, NF (STA-Rx) | 10 |
| Colloidal Silicon Dioxide, NF (Cab-O-Sil M5P) | 0.5 |
| Magnesium Stearate, NF | 0.6 |

The drug product may be stored at room temperature and is anticipated to be stable for at least 2 years at 25° C. and 50% RH. The drug is packaged in blister packs.

DIO-902 is administered to human subjects in a Phase I clinical trial to demonstrate that, at the therapeutically effective dose levels described herein, cardiolipin levels are increased.

All publications and patent documents (patents, published patent applications, and unpublished patent applications) cited herein are incorporated herein by reference as if each such publication or document was specifically and individually indicated to be incorporated herein by reference. Citation of publications and patent documents is not intended as an admission that any such document is pertinent prior art, nor does it constitute any admission as to the contents or date of the same. The invention, having been described in detail and exemplified above, has a wide variety of embodiments; consequently, while certain embodiments of the invention have been described herein in detail, numerous alternative embodiments are contemplated as falling within the scope of the following claims.

1. A method for treating a disease or condition associated with decreased cardiolipin levels or activity in a patient in need of such treatment, said method comprising administering a pharmaceutical composition comprising a therapeutically effective amount of 2S,4R ketoconazole enantiomer to said patient.

2. The method of claim 1, wherein said disease or condition is selected from the group consisting of Barth Syndrome, diabetic myopathy, cardiomyopathy associated with ageing, mitochondrial disease, and other conditions and disorders where cardiolipin deficiency is a component in a patient in need of such treatment.

3. The method of claim 2, wherein said disease or condition is Barth Syndrome.

4. The method of claim 2, wherein said disease or condition is diabetic cardiomyopathy

5. The method of claim 2, wherein said disease or condition is cardiomyopathy associated with ageing.

6. The method of claim 2, wherein said disease or condition is mitochondrial disease.

7. A method of delaying the onset of a disease or condition comprising administering a therapeutically effective amount of 2S,4R ketoconazole enantiomer to said patient,
wherein said disease or condition is selected from the group consisting of Barth Syndrome, diabetic myopathy cardiomyopathy associated with ageing, mitochondrial disease, and other conditions and disorders where cardiolipin deficiency is a component in a patient in need of such treatment.

8. A method of reducing the risk of developing a disease or condition comprising administering a therapeutically effective amount of 2S,4R ketoconazole enantiomer to said patient,
wherein said disease or condition is selected from the group consisting of Barth Syndrome, diabetic myopathy, cardiomyopathy associated with ageing, mitochondrial disease, and other conditions and disorders where cardiolipin deficiency is a component in a patient in need of such treatment.

9. A method of treating a disease or condition comprising co-administering to said patient a therapeutically effective amount of 2S,4R ketoconazole enantiomer and a compound selected from the group consisting of diuretics, adrenergic blocking agents, vasodilators, calcium channel blockers, renin inhibitors, angiotensin converting enzyme (ACE) inhibitors, angiotensin II antagonists, potassium channel activators, other cardiovascular agents, antioxidants, electron donors and acceptors, alternative energy sources, and agents that reduce lactate,
wherein said disease or condition is selected from the group consisting of Barth Syndrome, diabetic myopathy cardiomyopathy associated with ageing, mitochondrial disease, and other conditions and disorders where cardiolipin deficiency is a component in a patient in need of such treatment.

10. A method of claim 1, wherein the patient's cardiolipin level is determined before, during, and/or after administration of the 2S,4R ketoconazole enantiomer.

11. A pharmaceutical composition comprising 2S,4R ketoconazole enantiomer and a compound selected from the group consisting of diuretics, adrenergic blocking agents, vasodilators, calcium channel blockers, renin inhibitors, angiotensin converting enzyme (ACE) inhibitors, angiotensin II antagonists, potassium channel activators, other cardiovascular agents, antioxidants, electron donors and acceptors, alternative energy sources, and agents that reduce lactate.
